(19) United States
(12) Patent Application Publication
(10) Pub. No.: US 2009/0143830 A1
(43) Pub. Date: Jun. 4, 2009

(54) MOLDABLE PASTE COMPOSITION

(76) Inventors: Nicolas Bourgeois, Montreal (CA); Cameron M.L. Clokie, Toronto (CA); Uwe Tritthardt, Beaconsfield (CA); Michael McMillan, Saint-Lazare (CA)

Correspondence Address: Diane Dunn Mckay, Esq.;Mathews, Shepherd, McKay & Bruneau, P.A., Suite 201, 29 Thanet Road, Princeton, NJ, 08540, US

(21) Appl. No.: 12/235,703

(22) Filed: Sep. 23, 2008

(30) Foreign Application Priority Data
Mar. 23, 2007 (CA) PCT/CA2007/000476

(51) Int. Cl. A61F 5/00; A61L 27/50

(52) U.S. Cl. 606/86 R; 523/113

(57) ABSTRACT

The invention concerns a novel moldable and/or flowable composition for application to a bone defect site to promote new bone growth at the site. The composition includes a therapeutic material and a carrier comprising means for achieving reverse phase characteristics. In one embodiment, the therapeutic material can be a resorbable alloplastic material and the carrier can be a poloxamer. In a specific embodiment, the resorbable alloplastic material is a biphasic material composed of hydroxyapatite and tricalcium phosphate (HA-TCP), and the carrier is poloxamer 407 (Pluronic® F127). The invention further includes methods for using the novel composition.

### CROSS-REFERENCE TO RELATED APPLICATIONS

This application claims the benefit of U.S. Provisional Application No. 60/784,774 filed on Mar. 23, 2006, and International Patent Application PCT/CA2007/000476 filed on Mar. 23, 2007, which are hereby incorporated by reference in their entirety.

### FIELD OF THE INVENTION

The invention concerns a novel paste composition and its applications. More specifically, the present invention relates to a resorbable alloplastic composition that exhibits reverse phase behavior and is suitable for a number of applications, including without limitation reconstructive bone repair.

### BACKGROUND OF THE INVENTION

Osteoconductive bone repair materials are known in the art. Examples of such bone repair materials are described in U.S. Pat. No. 4,938,938 (Ewers et al.) and U.S. Pat. No. 6,881,227 (Jordanova-Spassova).

Bone repair materials can take several forms ranging from runny liquids to paste-like compositions. Both present advantages and disadvantages. Bone repair compositions that have a “runny” consistency are relatively easy to apply to and fill a bone defect well, but they may flow away from the repair site. In contrast to this, compositions that have a “paste-like” consistency are usually harder to apply to a defect but, once applied, tend to remain in place.

A common disadvantage with compositions that are currently available is that when they are placed in vivo, they warm up and their viscosity decreases (i.e., they tend to liquefy). The decrease in viscosity is due to the addition of thermal energy to the composition. The result is a composition that may not facilitate the containment of the bone grafting material, thus not optimizing the potential to promote bone growth and repair of the connective tissues.

More recently, biocompatible connective tissue compositions having reverse-phase characteristics have been developed. Examples of such compositions are described in U.S. Pat. Nos. 6,309,659 and 6,623,748 (Clokie).

Despite the existence of the above compositions, maxillofacial and orthopedic surgeons, amongst other health care professionals, still seek compositions that are biocompatible and readily applicable while being durable. Ideally, such compositions will also promote bone growth at a site of injury or repair. Accordingly, there is a need for a bone repair composition that is easy to apply to a defect site, that remains in place once applied to the defect site and that promotes bone growth.

The present invention seeks to meet this and related needs.

### SUMMARY OF THE INVENTION

Disclosed is a biocompatible composition to facilitate repair of connective tissues. The composition comprises a resorbable alloplastic material, and a carrier comprising a means for achieving reverse phase thermodynamic characteristics when mixed with the alloplastic material. The composition can be substantially liquid at 0° C., and substantially more viscous at 35° C., such that the composition has a consistency like that of a paste. The means for achieving reverse phase characteristics can comprise a block copolymer, such as a poly(oxyalkylene) block copolymer, which can be a poly(oxyethylene)-poly(oxypropylene)-poly(oxyethylene) triblock copolymer.

The means for achieving reverse phase characteristics comprises a poloxamer, such as poloxamer 407. The block copolymer can be a solid dispersed in a biocompatible solvent such as sterile water.

In an embodiment of the present invention, the carrier comprises a carrier of about 25 weight percent of a block copolymer dispersed in about 75 weight percent of a biocompatible solvent. To vary the consistency of the composition, the weight percentage of alloplastic material or other solid can be varied relative to the weight percentage of the carrier in the composition. For example, a paste-like form of the composition comprises about 50 weight percent of alloplastic material and about 50 weight percent of a carrier. A gel-like embodiment of the composition comprises about 40 weight percent of alloplastic material and about 60 weight percent of a carrier.

In a specific embodiment, the composition is comprised of a carrier consisting of a poly(oxyalkylene) block copolymer, water and a resorbable alloplastic material consisting of a biphasic material composed of hydroxyapatite and tricalcium phosphate.

The resorbable alloplastic material of the composition can comprise particles with a mean length of about 0.080-5.0 mm (80-5,000 microns), and a maximum diameter of about about 2.0 mm (2,000 microns).

Also disclosed is a method to facilitate the development of bone tissue, said method comprising: a moldable and/or flowable paste composition comprising alloplastic material, and, a carrier comprising a means for achieving reverse phase thermodynamic characteristics when mixed with the alloplastic material; and, placing the composition in a bony defect of a mammal.

### BRIEF DESCRIPTION OF THE FIGURES

FIG. 1: Aliquot containing a moldable paste composition;

FIG. 2: Rabbit cranium;

FIG. 3: Rabbit cranial vault defect preparation;

FIG. 4: Rabbit cranial vault defects;

FIG. 5: Example of grafted defects;

FIG. 6: Suturing of the scalp;

FIG. 7: Suturing completed;

FIG. 8: Harvested cranial graft sites (autogenous vs. non-grafted defect);

FIG. 9: Radiographic image of harvested cranium from FIG. 8;

FIG. 10: Harvested cranial graft sites (Product 1+Carrier vs. Product 1);

FIG. 11: Radiographic image of harvested cranium from FIG. 10;

FIG. 12: Histological image of harvested cranium from FIG. 10—cross section;

FIG. 13: Histological image of harvested cranium from FIG. 10—Product 1+Carrier side (×10);

FIG. 14: Histological image of harvested cranium from FIG. 10—Product 1+Carrier side (×40);

FIG. 15: Histological image of harvested cranium from FIG. 10—Product 1+side (×40);

FIG. 16: Histological image of harvested cranium from FIG. 10—Product 1 side (×40);

FIG. 17: Harvested cranial graft sites (Product 1+Carrier vs. Product 1+carrier+BMP);

FIG. 18: Radiographic image of harvested cranium from FIG. 17;

FIG. 19: Harvested cranial graft sites (Product 2 vs. Product 2+carrier);

FIG. 20: Radiographic image of harvested cranium from FIG. 19;

FIG. 21: Histological image of harvested cranium from FIG. 19—cross section;

FIG. 22: Histological image of harvested cranium from FIG. 19—Product 2 side (×10);

FIG. 23: Histological image of harvested cranium from FIG. 19—Product 2 side (×40);

FIG. 24: Histological image of harvested cranium from FIG. 19—Product 2+carrier side (×10);

FIG. 25: Histological image of harvested cranium from FIG. 19—Product 2+carrier side (×40);

FIG. 26: Harvested cranial graft sites (Product 2+BMP vs. Product 2+carrier+BMP);

FIG. 27: Radiographic image of harvested cranium from FIG. 26; and

FIG. 28: Percent Bone Fill in the Cranial Vault Defects.

FIG. 29: Bone regeneration in intra-femoral epiphysis bone defects filled with a composite of biphasic calcium phosphates (BCP) and Pluronic® F127.

### DESCRIPTION OF THE EMBODIMENTS

### Modes for Carrying Out Invention

### Definitions

Unless defined otherwise, all technical and scientific terms used herein have the same meanings as would be commonly understood by one of ordinary skill in the art to which this invention belongs. Although methods and materials similar or equivalent to those described herein can be used in the practice or testing of the present invention, the preferred methods, devices, and materials are now described.

All publications mentioned herein are incorporated by reference for the purpose of describing and disclosing the invention. The terminology used is for the purpose of describing particular embodiments only and is not intended to limit the scope of the present invention in any way.

Use of the singular forms “a,” “an,” and “the” include plural references unless the context clearly dictates otherwise.

As used in this specification and claim(s), the words “comprising” (and any form of comprising, such as “comprise” and “comprises” ), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

By “reverse phase” or “reverse thermal behavior” is intended a material that exhibits a physical property of becoming more viscous or solidified upon addition of thermal energy. It is believed that the solidification occurs by a mechanism other than that due to evaporation and corresponding loss of liquid.

As used herein, “ambient temperature” is 25° C., plus or minus 5° C.
As used herein, “body temperature” is 37° C., plus or minus 5° C.
As used herein, a “bony defect” or “bone defect site” is a bony environment of a mammal which comprises some viable bone tissue. The defect can be congenital, caused by trauma, or caused by disease.

“Osteoconductive” materials provide support for cells of a bone cell lineage, i.e., permitting cells of a bone cell lineage to grow along or through a matrix or lattice.

As used herein, the term “BMP” means bone morphogenetic protein. BMPs were “discovered” in the 1960s when a group of protein extracts were shown to help bones grow. In the 1980s, the proteins were individually identified and reproduced.

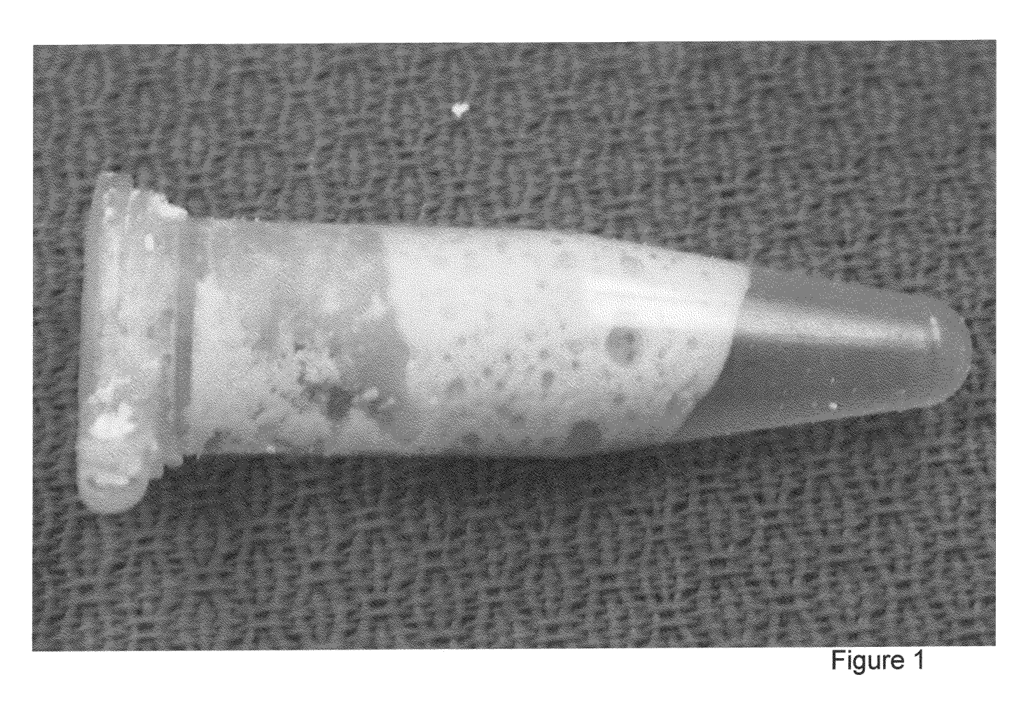

In one embodiment, the composition of the present invention is a flowable liquid when applied to a bony defect, whereupon the composition becomes increasingly solidified or viscous as it warms to ambient temperature and is further solidified as it warms to body temperature. (See FIG. 1, which shows an aliquot of the composition of the present invention in a microcentrifuge tube.) Upon being warmed (NB: “warmed” is okay here) to body temperature, the composition of the invention is a solid or highly viscous fluid. The reverse phase compositions in accordance with the invention are significantly different in principle from bone repair materials in the art, and do not function in the same way.

The composition comprises a therapeutic material for treating one or more connective tissue(s) and, a carrier. The therapeutic material can be a material to facilitate repair of connective tissues, i.e., a “connective tissue repair material.” The carrier achieves reverse phase characteristics when mixed with the therapeutic material.

The therapeutic material can be a material that is osteoconductive. The therapeutic material can be alloplastic. Ideally, this therapeutic material is resorbable.

Examples of alloplastic materials comprise hydroxyapatite (HA), tricalcium phosphate (TCP), beta-tricalcium phosphate (beta-TCP), biphasic material comprising of hydroxyapatite and tricalcium phosphate (biphasic calcium phosphates, or BCP), or combinations thereof. The resorbable alloplastic materials are plant derived or synthetically produced.

In a specific embodiment, the composition comprises a resorbable biphasic material (hydroxyapatite-tricalcium phosphate) in a carrier. This composition is suitable for application to a bone defect site to induce new bone growth. The composition of the present invention comprises the biphasic material hydroxyapatite (HA)-tricalcium phosphate (TCP) particles (referred to herein as “biphasic HA-TCP material”) in an inert biocompatible carrier.

The particles/granules have a mean length of about 0.080-5.0 mm (80-5,000 microns) and a maximum diameter of about 2.0 mm (2,000 microns).

The biocompatible carrier of the composition of the invention is a material that confers reverse phase thermodynamic properties on the composition. The use of Pluronic® F127 as a component of an osteointegration promoting composition is set forth in U.S. Pat. No. 5,503,558 (Clokie) and in International Patent Publication No. WO 95/13099. In a specific embodiment, the carrier comprises a polymer marketed by BASF (Parsippany, N.J.) as Pluronic® F127. Pluronic® F127 is a poly(oxyalkylene) block copolymer; more specifically, it is a poly(oxyethylene)-poly(oxypropylene)-poly(oxyethylene) poly(oxyethylene) triblock copolymer, which is a member of a class of compounds called poloxamers (Schmolka, “A Review of Block Polymer Surfactants” J. Am. Oil Chemists Soc. 54:110-116 (1977)). Several members of the poloxamer family exhibit reverse phase thermodynamic characteristics. Pluronic® F127 is also known by the name “poloxamer 407” (Schmolka, “A Comparison of Block Polymer Surfactant Gels” J. Am. Oil Chemist Soc. 68:206-209 (1991)). Pluronic® F127 has an average molecular weight of approximately 12,500 (Schmolka, “A Comparison of Block Polymer Surfactant Gels” J. Am. Oil Chemist Soc. 68:206 -209 (1991)).

In an embodiment of the composition of the present invention, the carrier is a liquid diluted in a solvent or is a solid dispersed in a solvent. In one embodiment, Pluronic® F127 is dispersed in a solvent such as sterile water. The Pluronic® F127 carrier is vastly different in size, molecular weight, and chemical structure than carriers in the art. The carrier is also substantially different in terms of its functional properties than any carrier of a bone repair material in the art.

The proposed composition has a unique physical property, being flowable at refrigerated temperatures and increasingly solidified at elevated temperatures, such as ambient and body temperatures. This property is referred to in the art as “reverse phase” or “reverse thermal behavior”. Due to the reverse phase property of the proposed composition, the composition is generally manufactured at refrigerated temperatures, such as 5° C. Manufacturing is done at refrigerated temperatures to enhance mixing of the components of the composition, since the proposed composition comprising an aqueous suspension of Pluronic® F127 begins to become more viscous at ambient temperature, and is increasingly viscous and solidified at body temperature. Generally, a composition of the invention will be twice as viscous at 35° C, as it is at 0° C.

For example, the Pluronic® F127 carrier in the composition of the present invention (when dispersed in an appropriate amount of sterile water), has the unique property of being a liquid at refrigerated temperature and increasingly solidified, then solid at elevated temperature, absent the effects of evaporation and concomitant loss of water. This property is called “reverse phase” or “reverse thermal behavior” because it is the exact opposite of the thermodynamic properties exhibited by standard carriers.

It is believed that the reverse phase property is due, at least in part, to the fact that Pluronic® F127 is composed of discrete blocks of both hydrophilic (i.e., oxyethylene) and hydrophobic (i.e., oxypropylene) subunits. (See, for example, Schmolka, “A Comparison of Block Polymer Surfactant Gels” J. Am. Oil Chemist Soc. 68:206-209 (1991).)

In contrast, standard carriers, as well as all liquids, manifest the typical physical property of becoming increasingly flowable upon addition of thermal energy, such as occurs when the liquid is heated to body temperature. However, the carrier in a composition of the present invention becomes less flowable as energy is added to it either by heating or by shaking.

The unique reverse phase thermodynamic properties of the composition of the present invention allow the product to function in a substantially different, and more convenient, manner relative to other flowable bone repair products. When applied to a bone defect site, the reverse phase property of the carrier provides support characteristics for the composition that are substantially different than the characteristics of standard carriers. Enhanced support is provided by the composition of the invention. The Pluronic® F127 carrier of the composition of the present invention helps to provide support characteristics which are unlike those of any standard carrier. This is because the composition is flowable at refrigerated temperature and can thus readily be applied to a bony defect site, but it becomes increasingly viscous and solidifies upon warming at the site. The solidification of the composition of the present invention achieves several beneficial effects. When solidified, the composition does not flow away from the defect site, and the solidified product immediately augments and facilitates structural support at the defect. Also, since the osteogenic composition of the invention is initially liquid, it readily fills a defect, then becomes solidified and achieves enhanced osteogenesis. Moreover, with compositions of the invention, comprising a sterile aqueous colloidal suspension of Pluronic® F127 as a carrier and resorbable biphasic HA-TCP material, the carrier will resorb or dissolve after about three days, leaving the osteoconductive alloplastic material at the bone defect site. It is believed to be advantageous that the carrier disperses as this then allows enhanced ingrowth of connective or vascular tissues.

In a composition of the invention, the weight percentages of the alloplastic material and the carrier can each be varied. For example, the weight percent of the resorbable alloplastic material can vary between about 20 to 80 weight percent of the composition, and the weight percent of the carrier can vary between about 20 to 80 weight percent of the composition.

### EXAMPLE 1

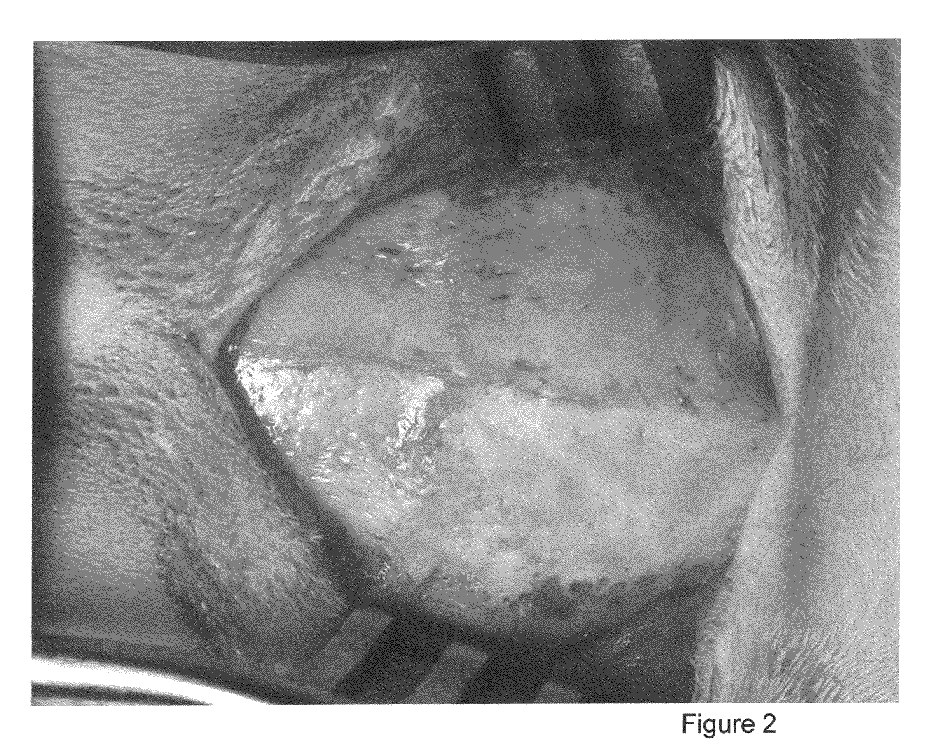

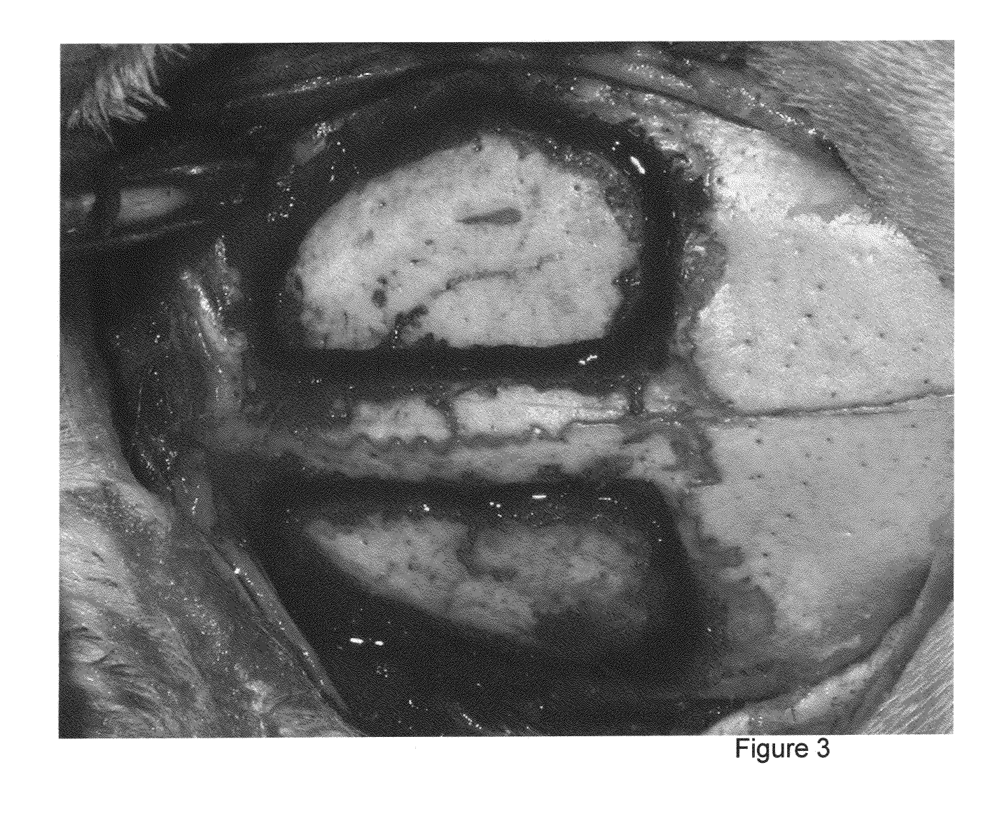

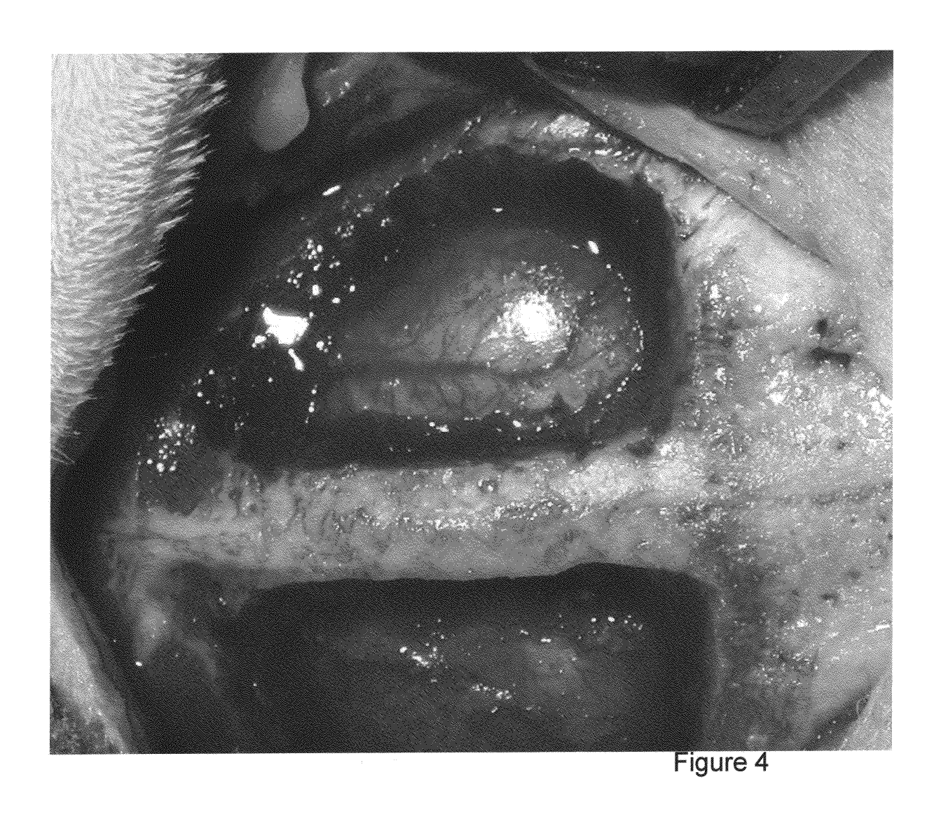

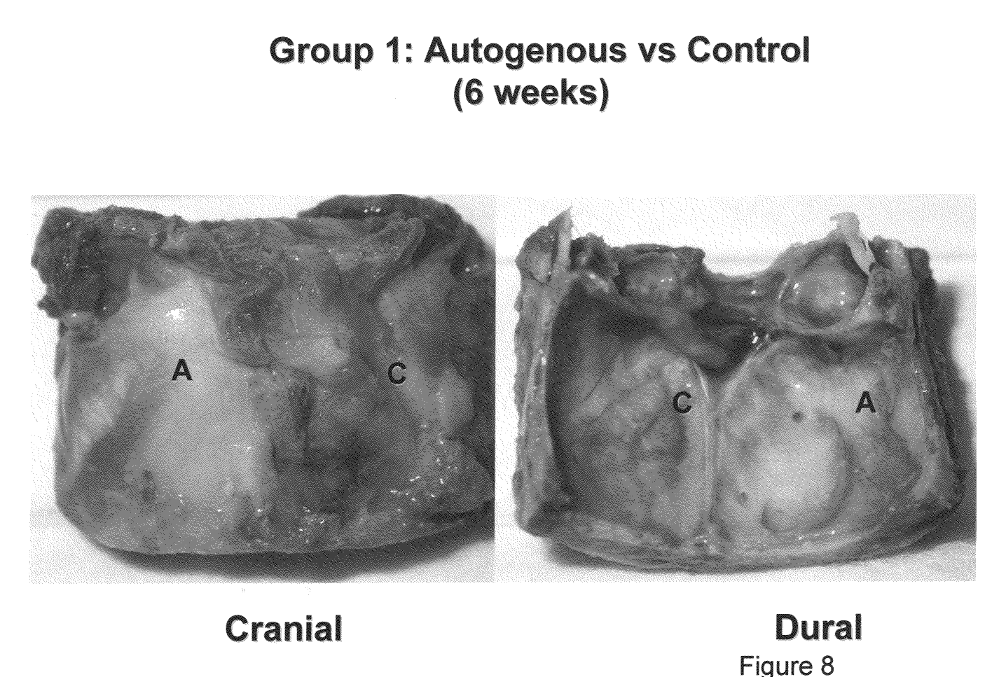

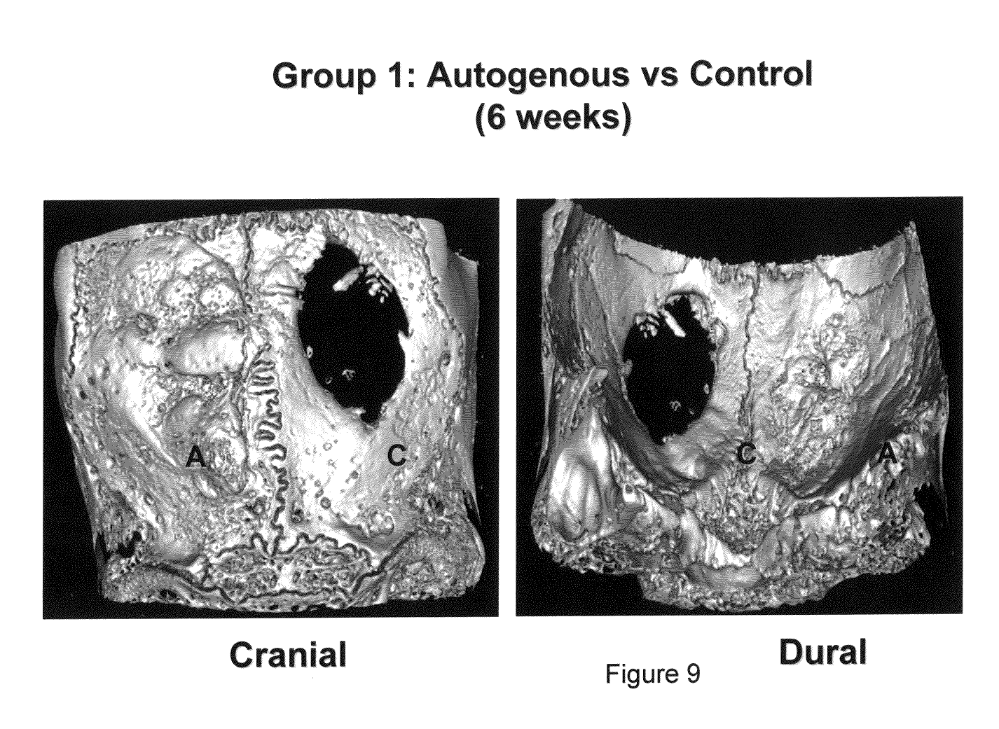

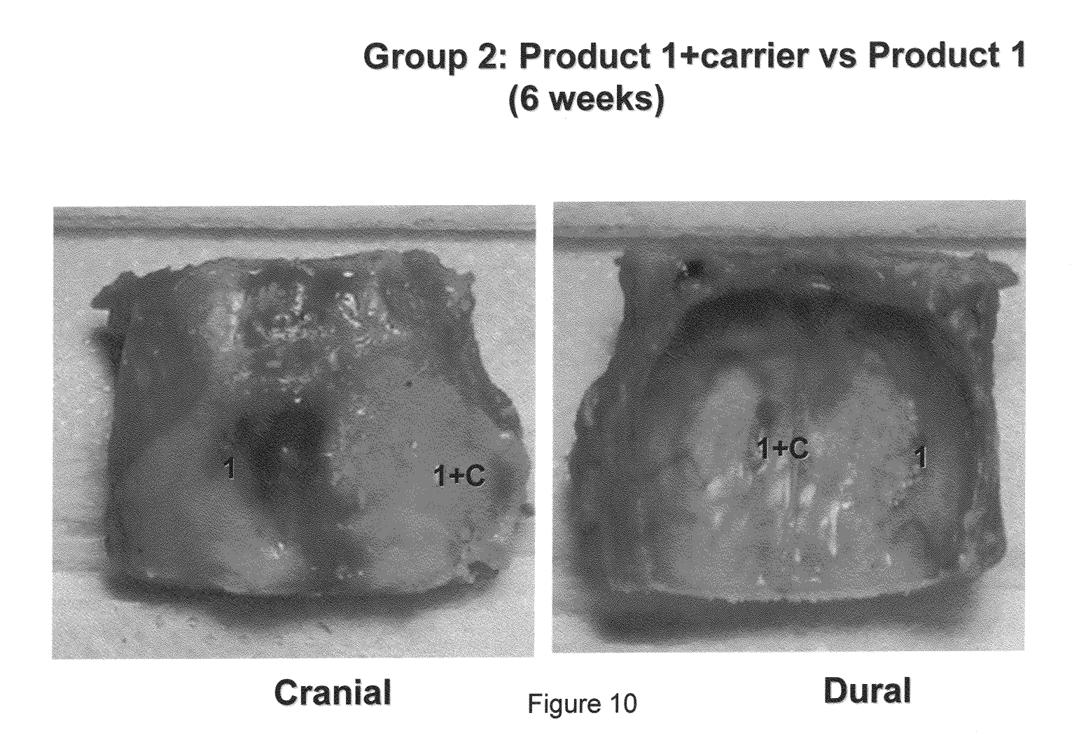

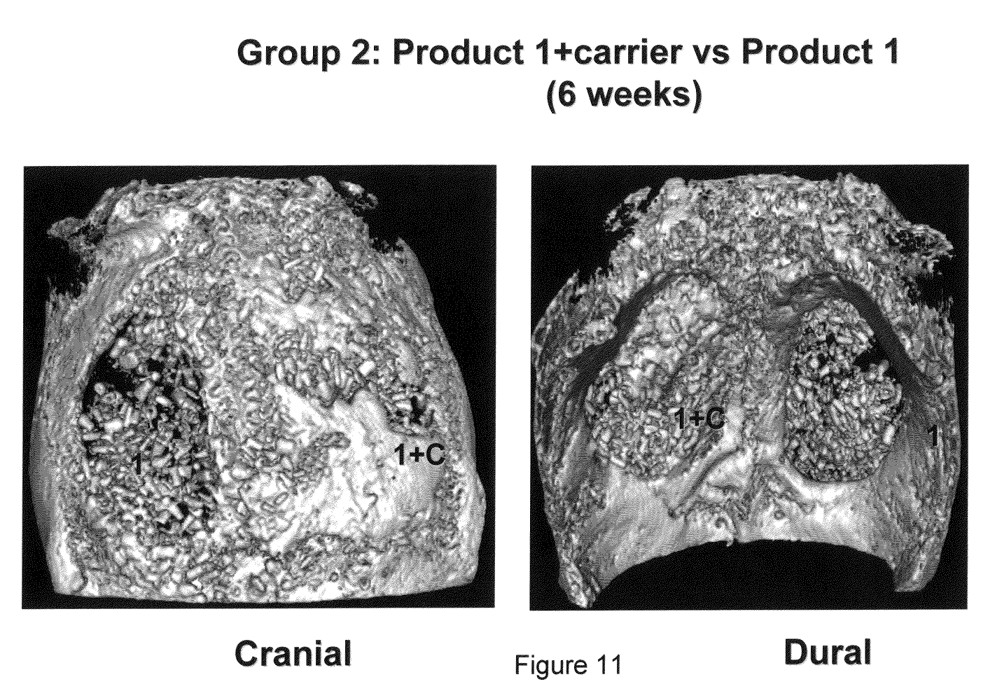

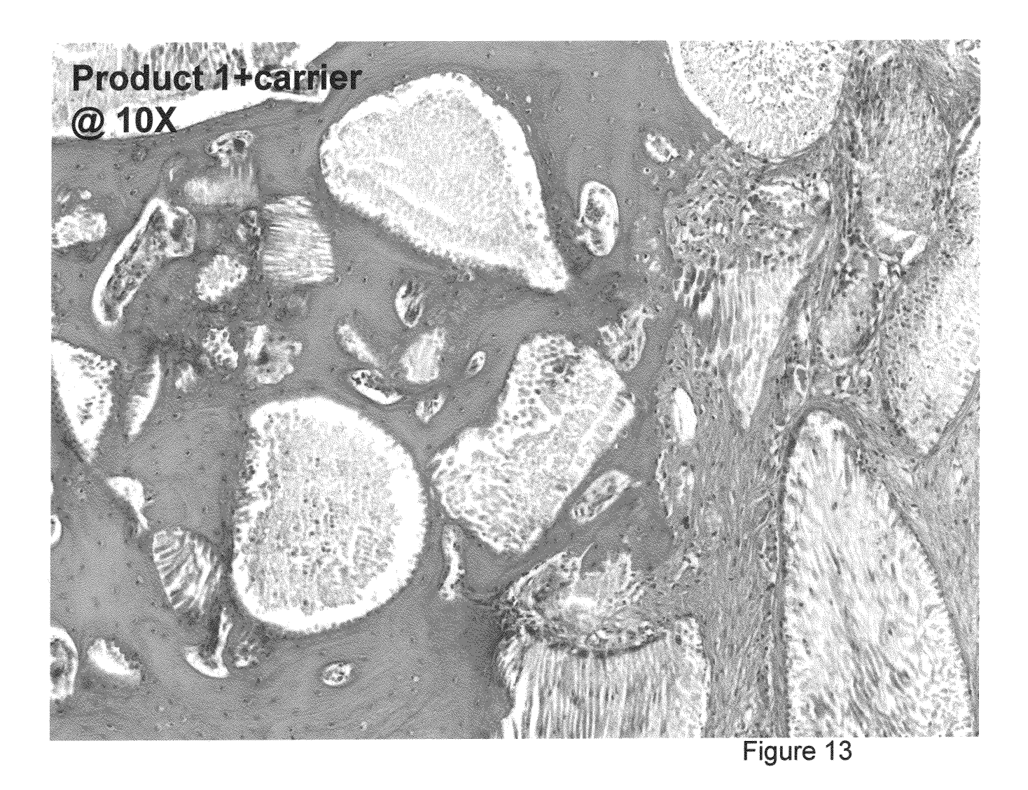

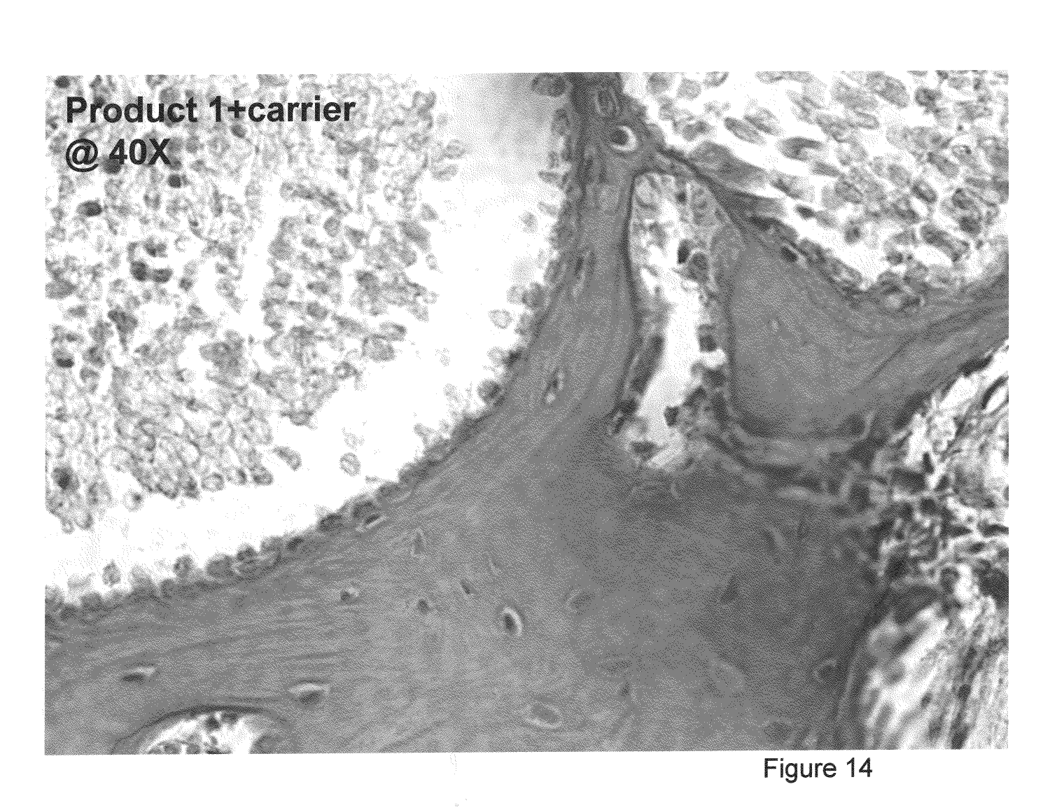

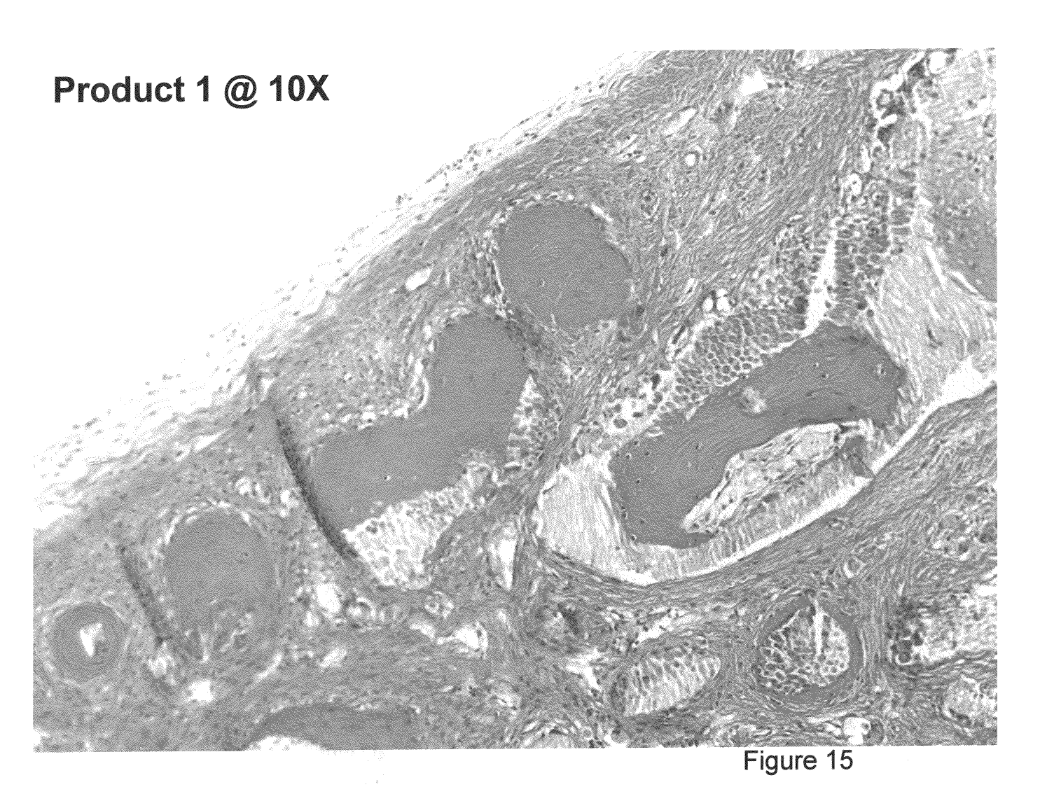

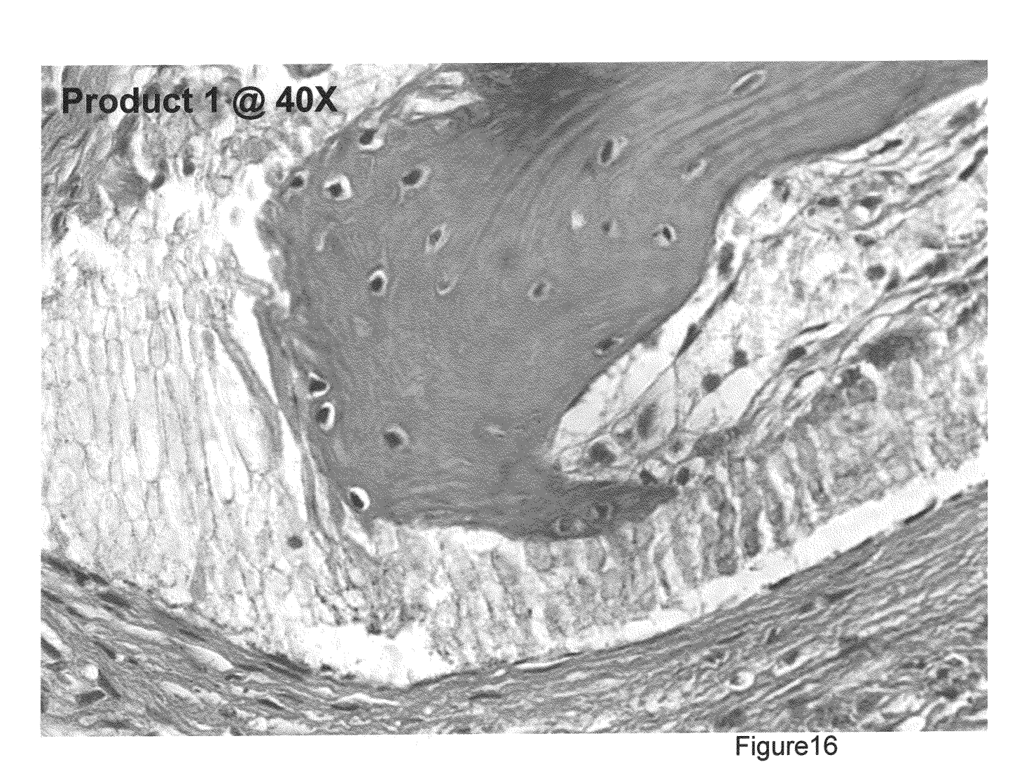

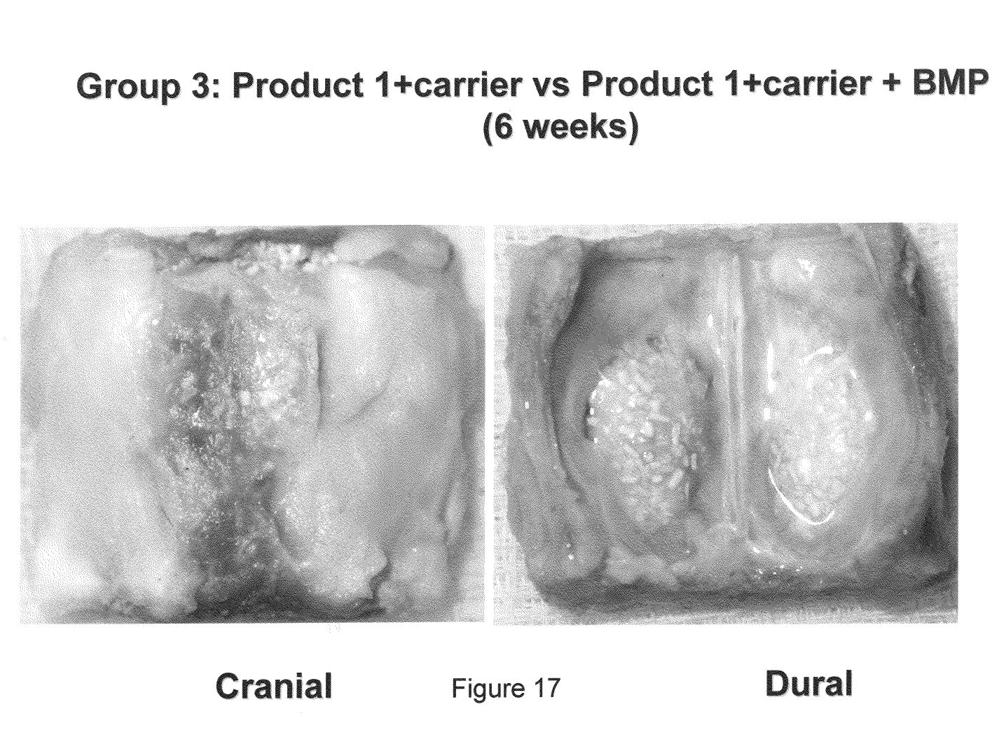

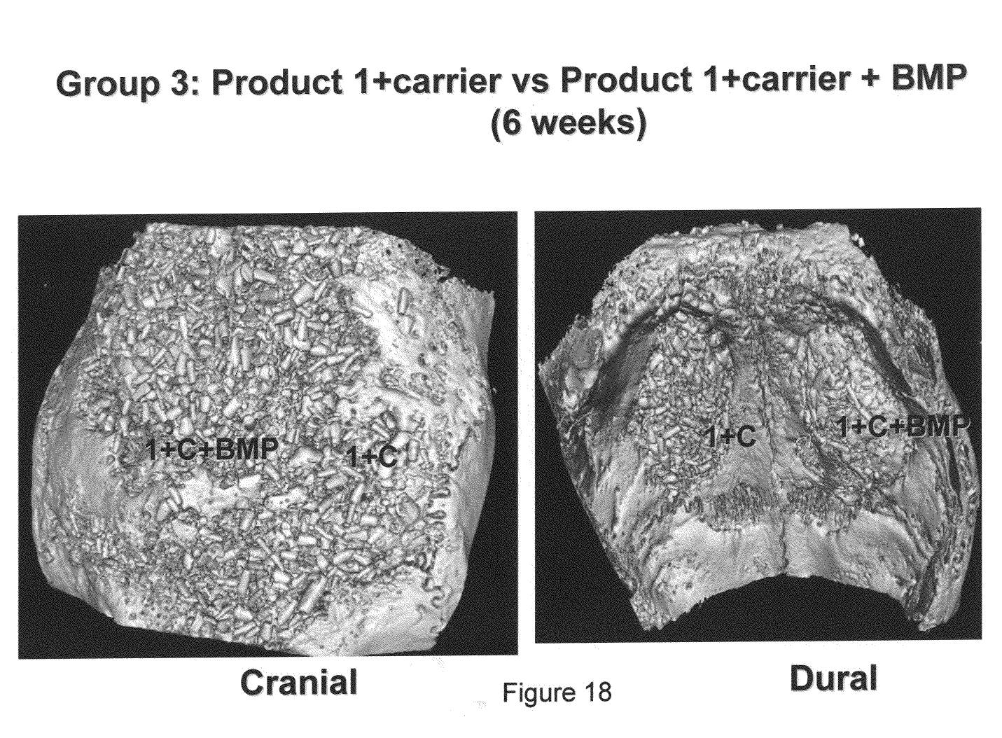

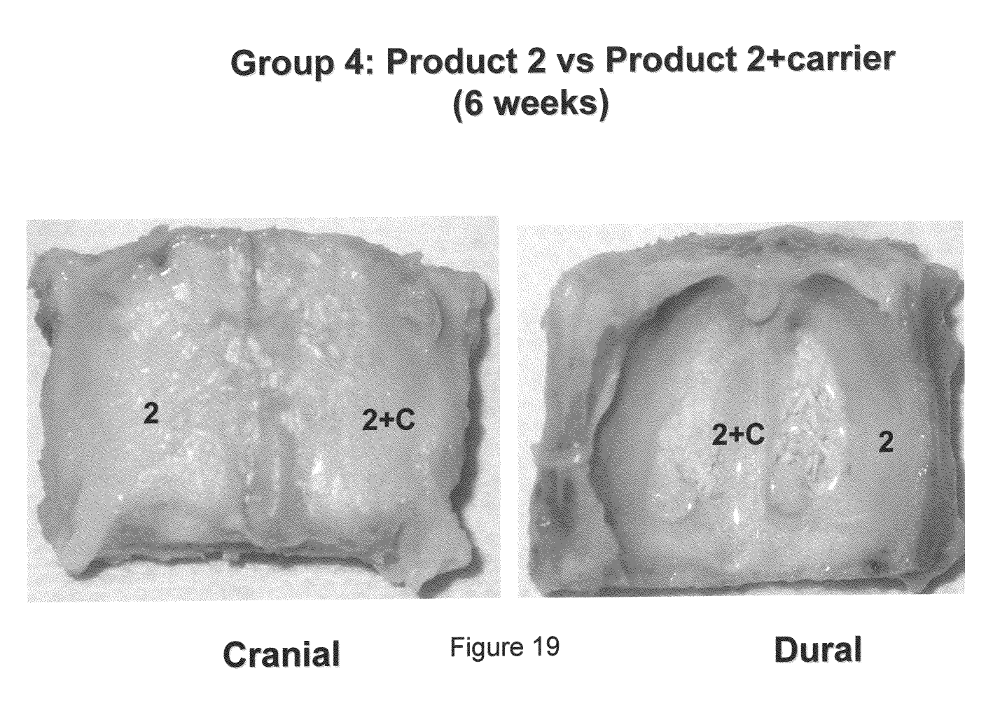

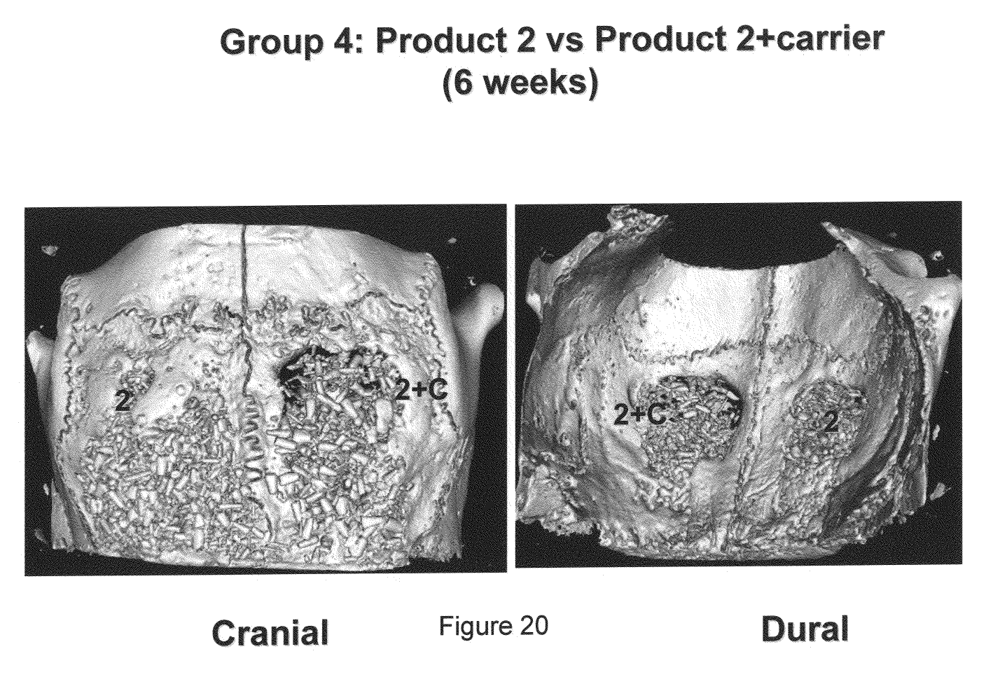

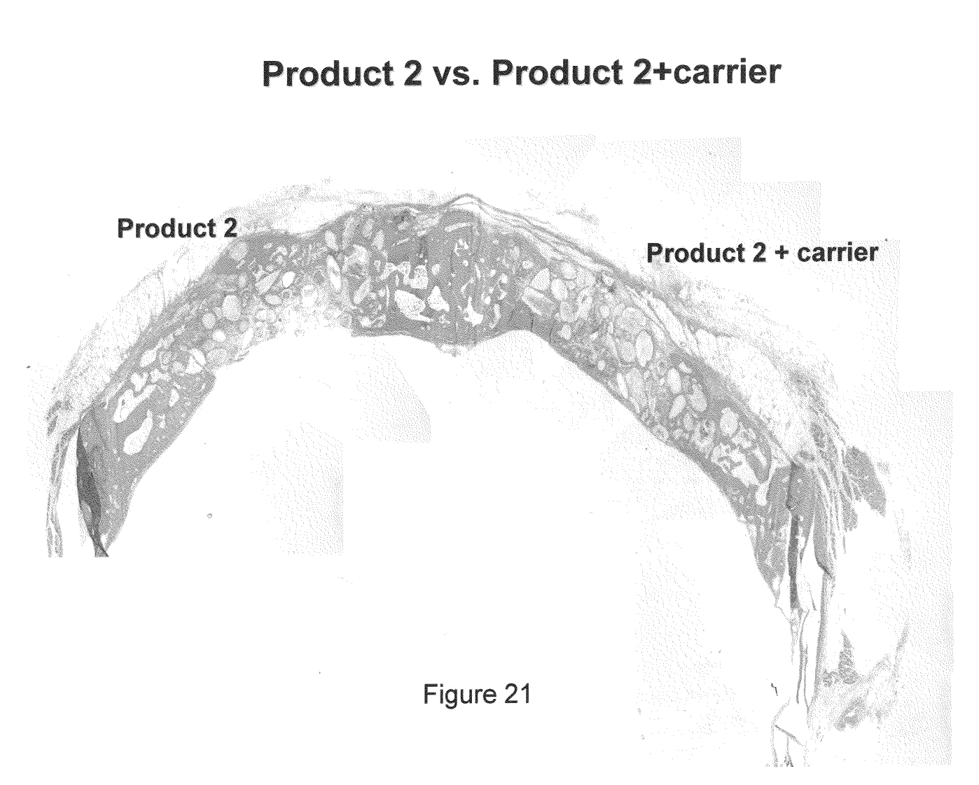

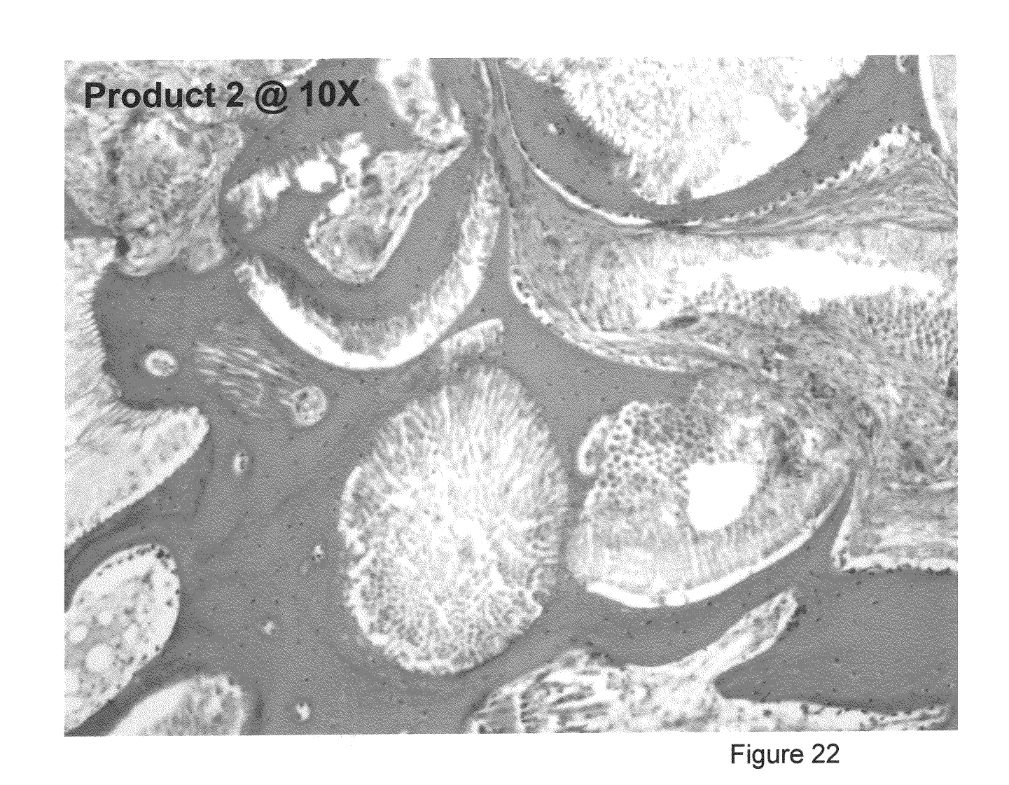

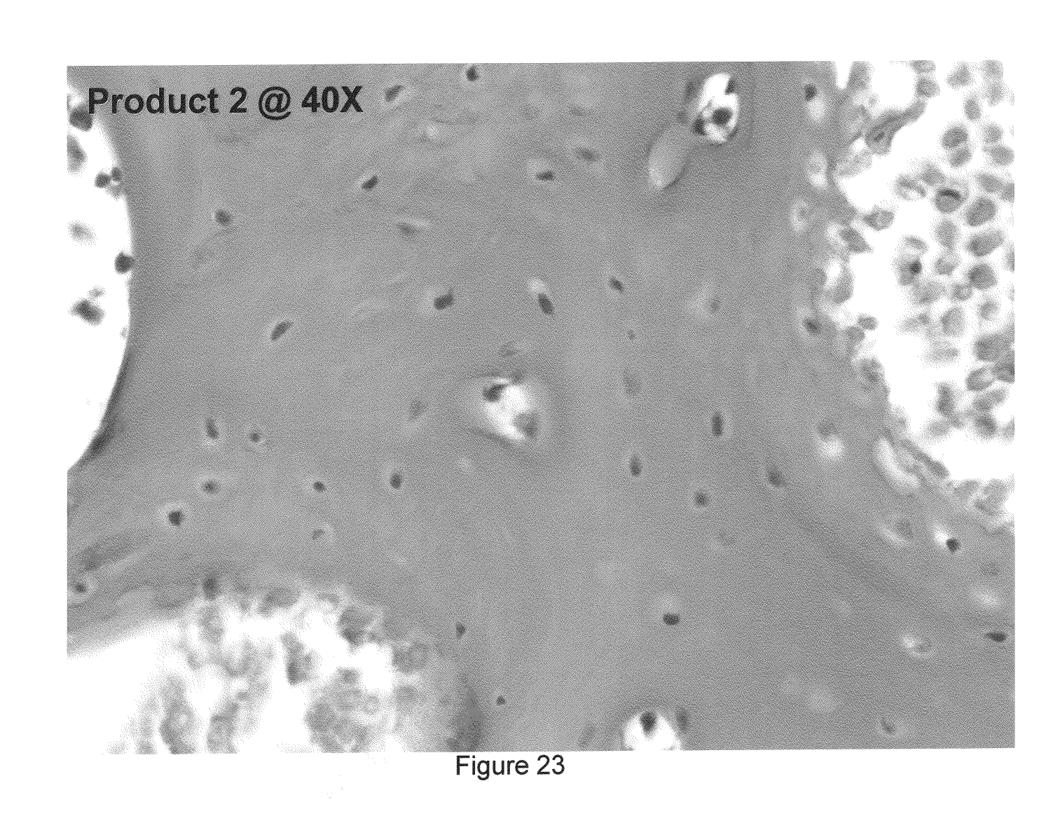

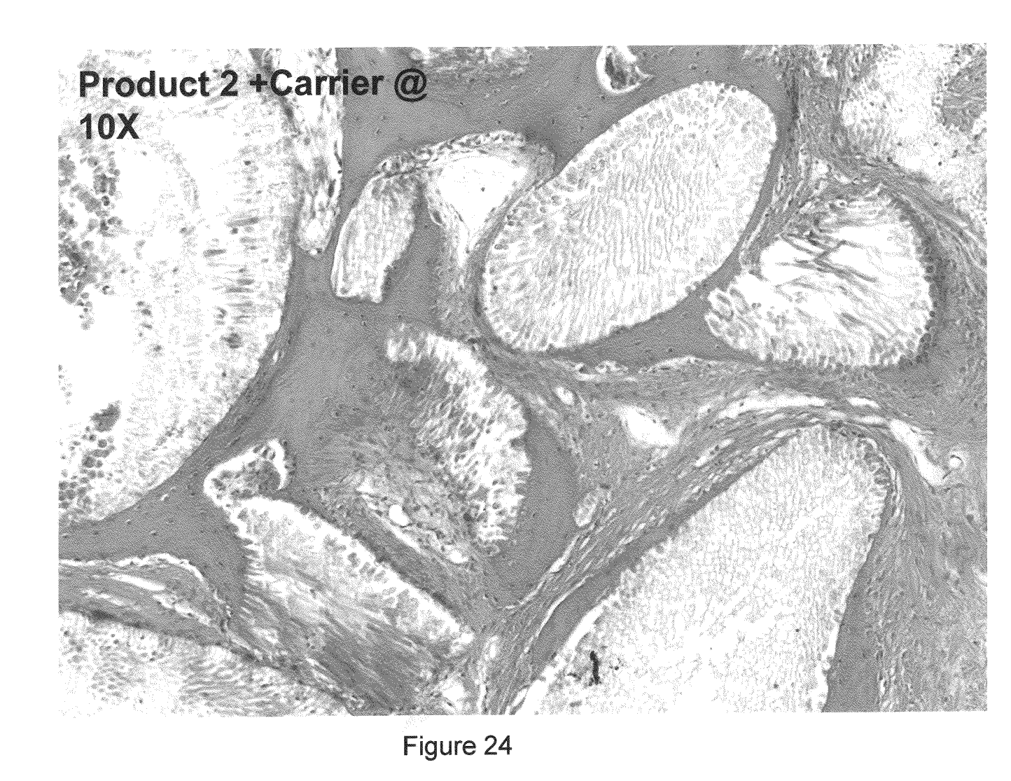

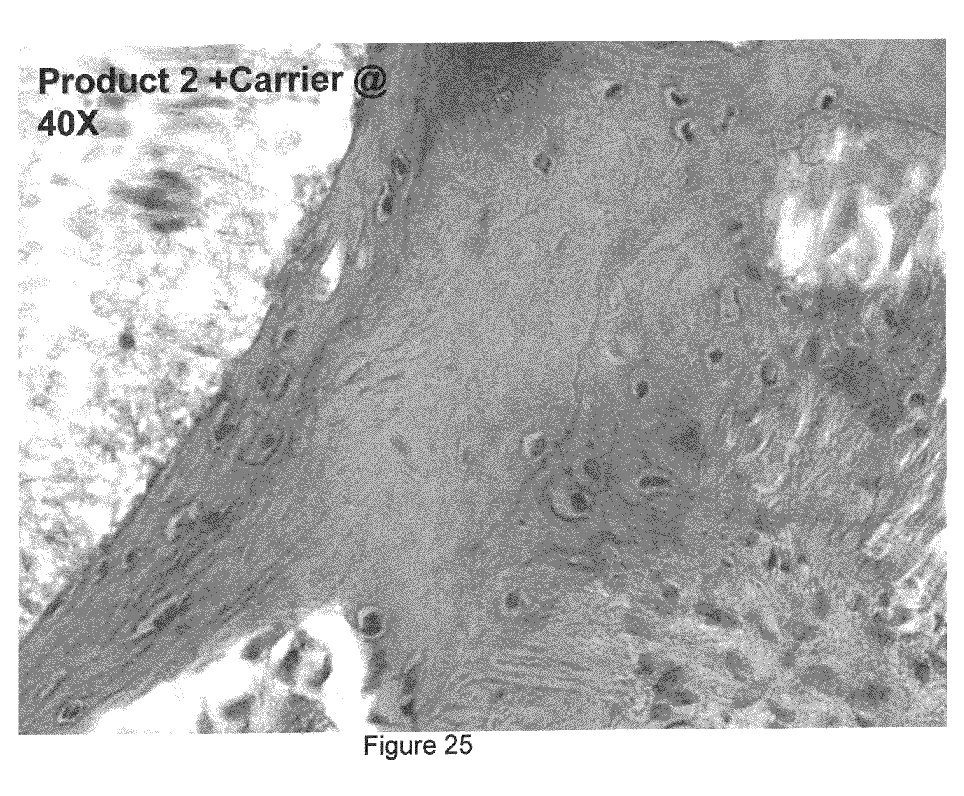

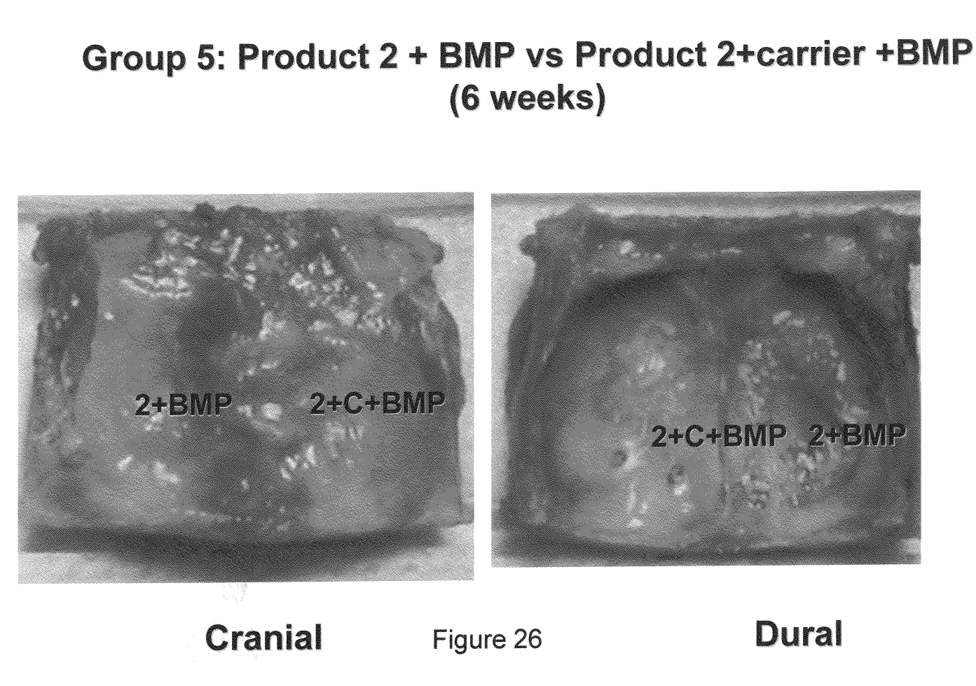

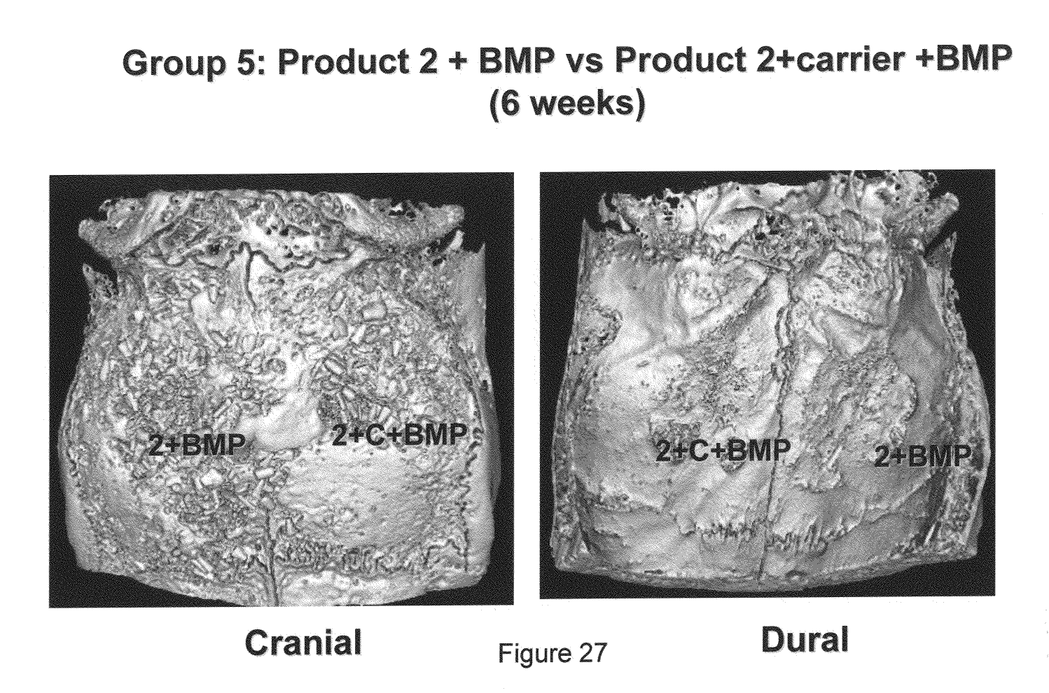

### Experimental Design:

| | |
|---|---|
| Animals: | 50 New Zealand White male rabbits weighing 3.5-4.0 kg were |
| | divided into 5 groups (n = 10) and were sacrificed at 6 |
| | weeks. Bilateral 15 mm diameter critical sized defects were |
| | created in the parietal bones of each animal (FIGS. 2 to 4). |
| Groups: | 1) The first group (n = 10) had defects in one side left |
| | unfilled. The other side was filled with autogenous |
| | particulate bone harvested from the cranium (FIGS. 8 and 9). |
| | 2) The second group (n = 10) had defects in one side |
| | filled with Product 1 while the other side were filled with |
| | Product 1 + carrier (FIGS. 10 to 16). |
| | 3) The third group (n = 10) had defects in one side will |
| | be filled with Product 1 + carrier. The other side was |
| | filled with Product 1 + carrier + BMP (FIGS. 17 and 18). |
| | 4) The fourth group (n = 10) had defects in one side will |
| | be filled with Product 2. The other side was filled with |
| | Product 2 + carrier (FIGS. 19 to 25). |
| | 5) The fifth group (n = 10) were defects in one side will |
| | be filled with Product 2 + BMP. The other side was filled |
| | with Product 2 + carrier + BMP (FIGS. 26 and 27). |

### Surgery:

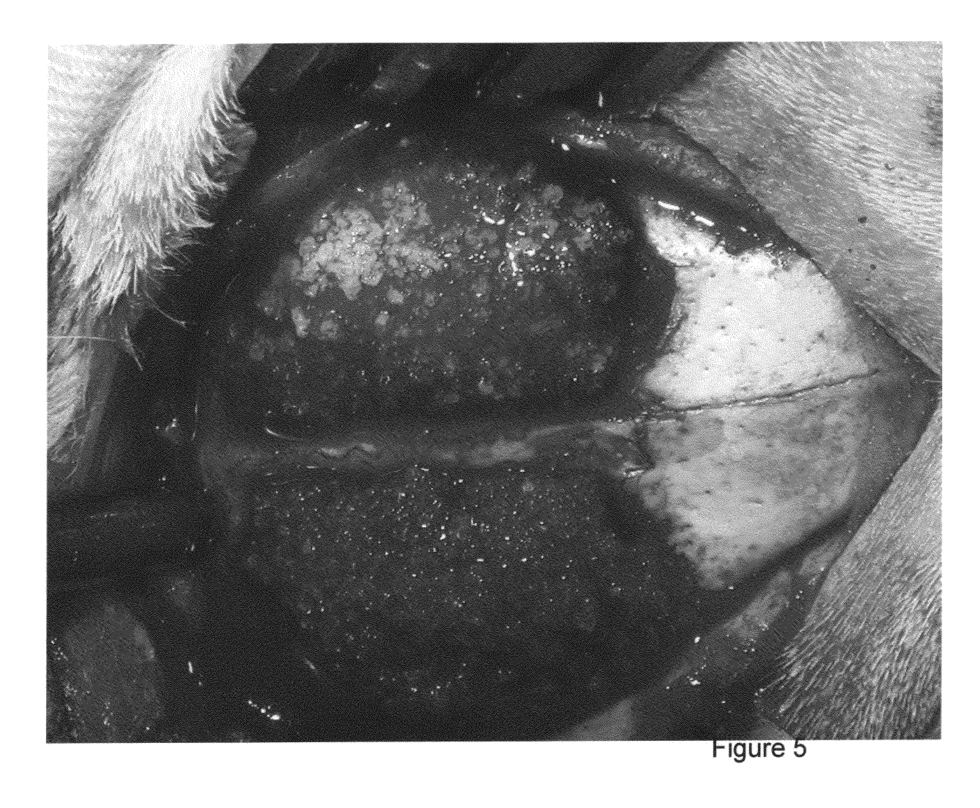

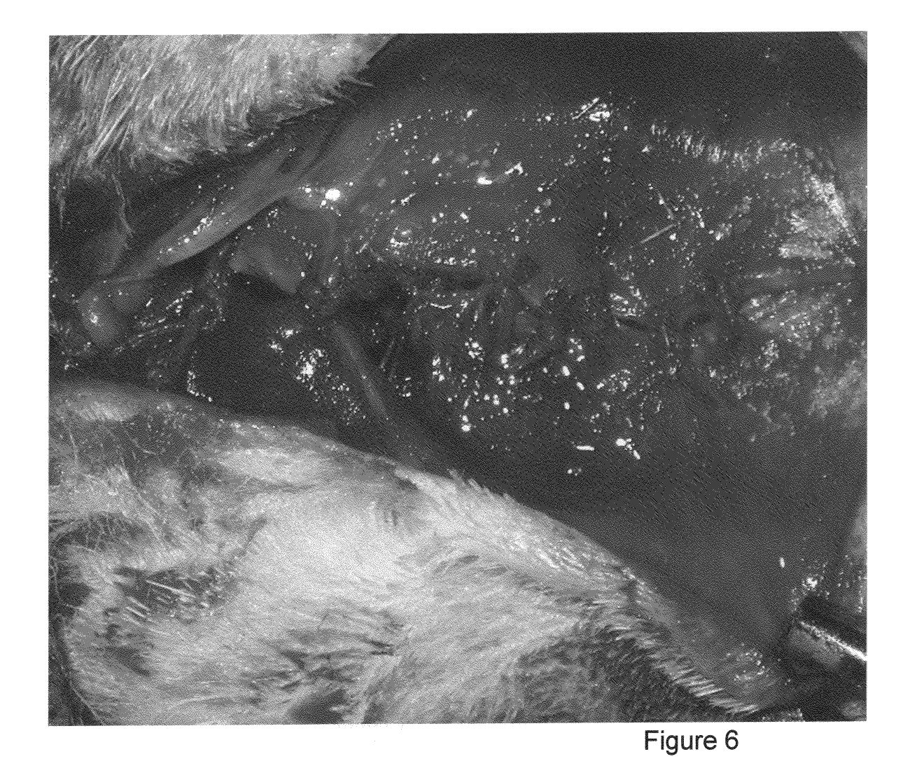

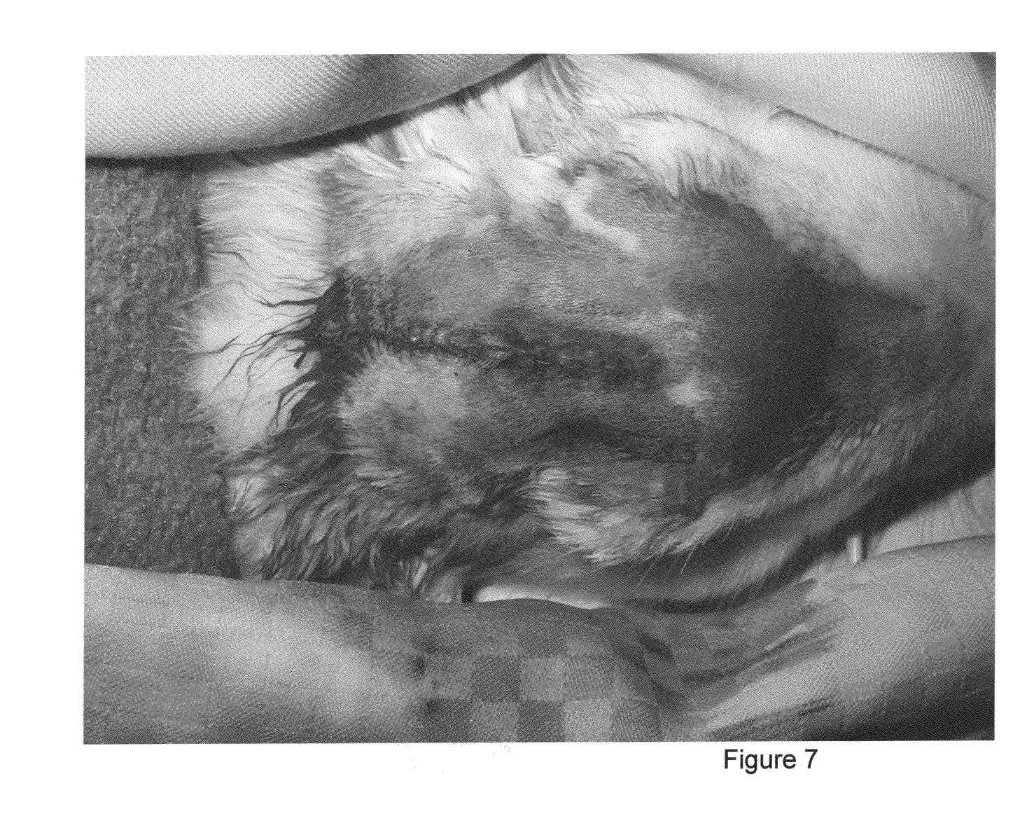

The surgical procedures for this investigation were performed according to recognized techniques approved by the University of Toronto, Animal Care Committee. The surgical procedures were performed using aseptic techniques. Each animal had two critical sized defects created in its parietal bone bilaterally. Grafting material was randomly allocated to each defect (FIGS. 2 to 7).

### Radiographic Analysis:

Plain film radiographs for parietal bones were taken with a Cephalostat machine. Micro CT images were also taken with Explore Locus SPA micro CT scanner (GE medical systems) (FIGS. 9, 11, 18, 20, and 27).

### Histological Analysis:

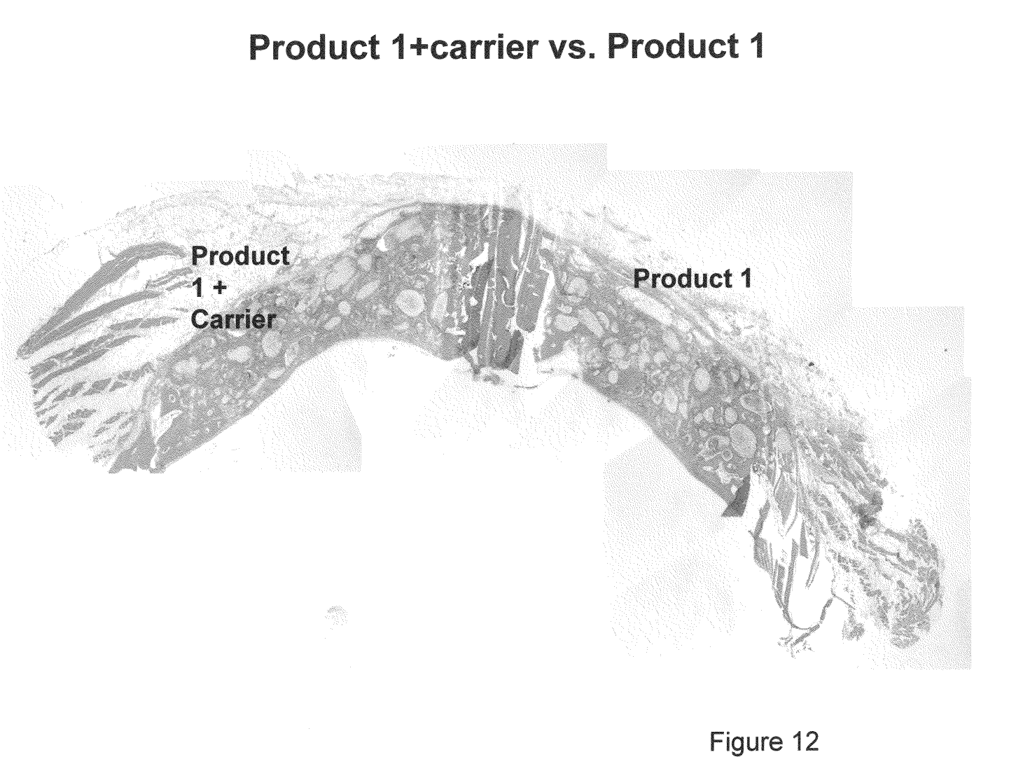

Following radiographic evaluation, the tissue samples were fixed, sectioned, stained and prepared for analysis. This included a morphometric evaluation of hematoxylin-eosin stained light microscopic sections using an image analysis software package (Image Pro Plus). Morphological parameters including the relative amounts of bone, bone marrow, blood vessels were evaluated statistically using ANOVA (FIGS. 12 to 16 and 21 to 25).

### Results:

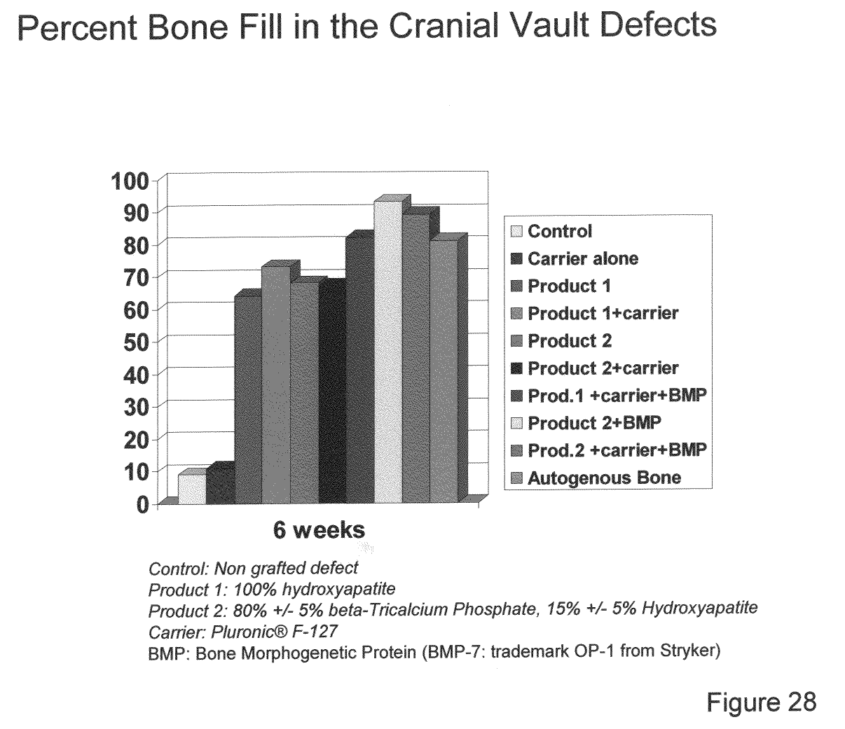

An analysis of the histomorphometric results of the data derived from the careful evaluation of the samples revealed significant differences (p<0.05) between negative control groups and all other groups tested with the negative control groups demonstrating significantly less bone within the defects than the other groups being evaluated. (See FIG. 28.) Further differences were identified between groups treated with BMP including the autogenous bone group and those untreated with BMP. In this case those treated with BMP demonstrated significantly (p<0.05) more new bone than those that were left untreated. Of note there were no differences between the autogenous bone group and those treated with BMP and there were no differences between groups where the carrier was used and those where it was not. The results of the various imaging studies support the histomorphometric findings (FIG. 28).

### EXAMPLE 2

The aim of this study was to develop and to test in vivo handling, setting and osteogenic properties of injectable bone substitute materials by combining specific granules of BCP with or without radiopaque elements with a thermo sensitive resorbable inert carrier such as Pluronic® F127 (Pluronic, BASF, Mt. Olive, N.J.) [2]. The composite is liquid at ambient temperature and set as hydrogel at 37° C.

### Experimental Design:

Three different rounded BCP granules bioceramics were prepared in the range of 80-200 μm. Rounded granules were prepared from calcium deficient apatite (CDA) with or without Barium sulphate radio-opaque. The sintering involved crystallization of BCP with different HA/TCP ratios (60/40 and 20/80). Sixty percent (60%) in weight of BCP granules were mixed with Pluronic®F127 (A: 60/40 BCP, B: 20/80, C: 60/40 with Barium). Fifteen (15) New Zealand rabbits were used. Bilateral 6 mm intra-femoral epiphysis bone defects were totally filled by composite. Lumbar muscular implantation (1 cc) were bilaterally implanted. After 3 and 6 weeks, the explants were fixed in a solution of neutral formalin solution, dehydrated and embedded in GMA. Micro CT was realized for 3D reconstruction. Thin sections were stained using Movat's pentachrome. Thicker sections realized with a diamond saw microtome were examined in SEM using BSE and image analysis. Muscular implants were prepared for histology, after paraffin embedding, sections were stained by hematoxylin eosin. Bone regeneration and BCP resorption were evaluated quantitatively by histomorphometry Statistics were performed using Student's t-test.

### Results:

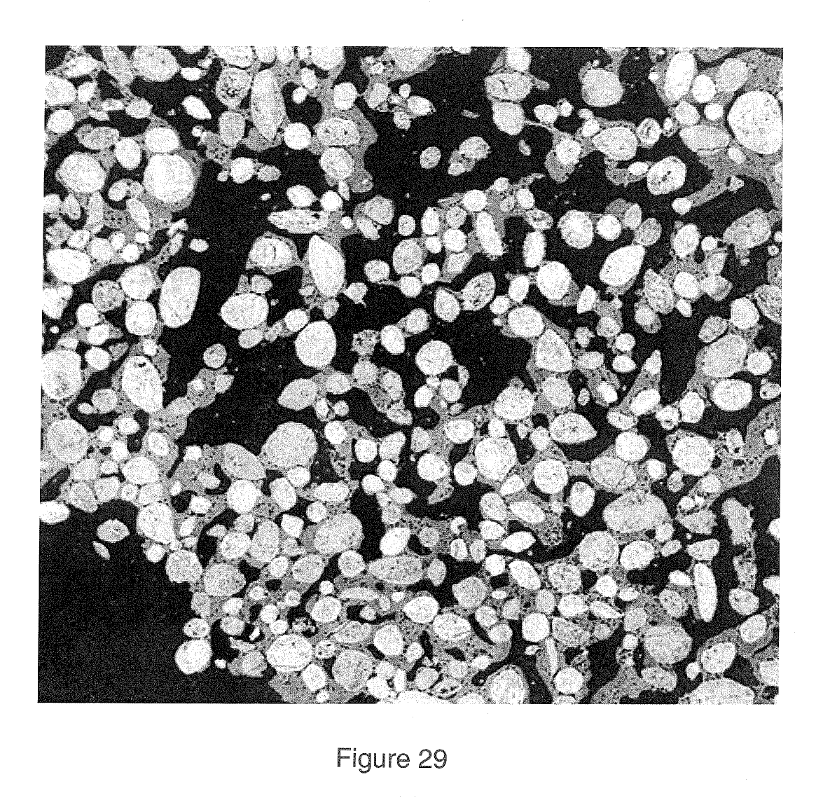

Bone regeneration was similar over the time period studied. From 3 to 6 weeks lamellar bone trabeculae appeared at the expense of both composites. BCP granules play the role of scaffolds for osteoconduction. Radio opaque composite have same bone ingrowth than the two others samples BCP. Newly-formed bone was observed mainly in deep zones of defects from the surface to the core. (See FIG. 29.) Thermosensitive polymer increased the handling and the moldability without compromising BCP osteoconductivity. Intramuscular implantations confirm the biocompatibility.

These results suggest that Pluronic® F127 can be used to enhance handling and moldability without any negative effects on the osteogenecity of BCP specific granules.

Although the present invention has been described hereinabove by way of exemplified embodiments thereof, it can be modified without departing from the spirit, scope and nature of the subject invention, as defined in the appended claims.

What is claimed is:

1. A moldable and/or flowable paste, comprising:
an osteoconductive resorbable alloplastic material; and
a mixture of a poly(oxyalkylene) block copolymer and water that exhibits reverse phase behavior when its temperature is increased from ambient to body temperature.

2. The composition of claim 1, wherein the poly(oxyalkylene) block copolymer is Pluronic® F127.

3. The composition of claim 1, wherein the mixture of poly(oxyalkylene) block copolymer and water is about 25 weight percent poly(oxyalkylene) block copolymer and about 75 weight percent water.

4. The composition of claim 1, wherein said paste comprises between about 40 to 50 weight percent alloplastic material and about 50 to 60 weight percent poly(oxyalkylene) block copolymer and water.

5. The composition of claim 1, wherein the resorbable alloplastic material is plant derived or synthetically produced.

6. The composition of claim 1, wherein the resorbable alloplastic material is chosen from the group consisting of hydroxyapatite (HA), tricalcium phosphate (TCP), beta-tricalcium phosphate (beta-TCP) and biphasic calcium phosphates (BCP).

7. The composition of claim 1, wherein the resorbable alloplastic material is a biphasic material composed of HA and beta-TCP.

8. The composition of claim 7, wherein the resorbable alloplastic material is comprised of beta-TCP having a content of between about 40 to 90 weight percent and HA having a content of about between 10 and 60 weight percent.

9. The composition of claim 8, wherein the proportion of BCP mixed with Pluronic® F127 is chosen from the group consisting of the following HA/beta-TCP ratios: 60/40, 20/80 and 60/40 with Barium.

10. The composition of claim 8, wherein said composition is comprised of particles/granules having a mean length of about 0.080-5.0 mm (80-5,000 microns) and a maximum diameter of about 2.0 mm (2,000 microns).

11. A method to facilitate the development of bone tissue comprising:
placing a moldable and/or flowable composition at a bone defect site, wherein said composition comprises, an osteoconductive resorbable alloplastic material, and
a mixture of a poly(oxyalkylene) block copolymer and water that exhibits reverse phase behavior when its temperature is increased from ambient to body temperature.

12. A method as defined in claim 11, wherein said composition is as defined in claim 2.

13. A method as defined in claim 11, wherein said composition is as defined in claim 3.

14. A method as defined in claim 11, wherein said composition is as defined in claim 4.

15. A method as defined in claim 11, wherein said composition is as defined in claim 5.

16. A method as defined in claim 11, wherein said composition is as defined in claim 6.

17. A method as defined in claim 11, wherein said composition is as defined in claim 7.

18. A method as defined in claim 11, wherein said composition is as defined in claim 8.

19. A method as defined in claim 11, wherein said composition is as defined in claim 9.

20. A method as defined in claim 11, wherein said composition is as defined in claim 10.
